(19) United States
(12) Patent Application Publication
(10) Pub. No.: US 2012/0308562 A1
(43) Pub. Date: Dec. 6, 2012

(54) METHODS OF TREATING MESOTHELIOMA WITH A PI3K INHIBITOR COMPOUND

(76) Inventors: Mika K. Derynck, San Mateo, CA (US); Jennifer O'Hara Lauchle, Larkspur, CA (US)

(21) Appl. No.: 13/486,141

(22) Filed: Jun. 1, 2012

Related U.S. Application Data
(60) Provisional application No. 61/492,966, filed on Jun. 3, 2011.

(51) Int. Cl. A61K 31/5377; A61K 31/7068; A61K 33/24; A61P 35/00; A61K 39/395

(52) U.S. Cl. 424/133.1; 514/234.2; 424/649; 514/49

(57) ABSTRACT

Methods are provided for treating mesothelioma patients with a dual PI3K/mTOR inhibitor, GDC-0980: (S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one, and having the structure:

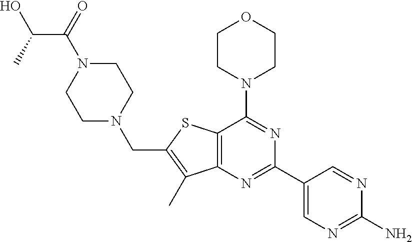

### CROSS REFERENCE TO RELATED APPLICATIONS

This non-provisional application filed under 37 CFR §1.53(b), claims the benefit under 35 USC §119(e) of U.S. Provisional Application Ser. No. 61/492,966 filed on 3 Jun. 2011, which is incorporated by reference in entirety.

### FIELD OF THE INVENTION

The invention relates to methods of using a PI3K inhibitor compound for the treatment of mesothelioma.

### BACKGROUND OF THE INVENTION

Phosphoinositide 3-kinases (PI3K) are lipid kinases that phosphorylate lipids at the 3-hydroxyl residue of an inositol ring (Whitman et al (1988) Nature, 332:664). The 3-phosphorylated phospholipids (PIP3s) generated by PI3-kinases act as second messengers recruiting kinases with lipid binding domains (including plekstrin homology (PH) regions), such as Akt and phosphoinositide-dependent kinase-1 (PDK1). Binding of Akt to membrane PIP3s causes the translocation of Akt to the plasma membrane, bringing Akt into contact with PDK1, which is responsible for activating Akt. The tumor-suppressor phosphatase, PTEN, dephosphorylates PIP3 and therefore acts as a negative regulator of Akt activation. The PI3-kinases Akt and PDK1 are important in the regulation of many cellular processes including cell cycle regulation, proliferation, survival, apoptosis and motility and are significant components of the molecular mechanisms of diseases such as cancer, diabetes and immune inflammation (Vivanco et al (2002) Nature Rev. Cancer 2:489; Phillips et al (1998) Cancer 83:41).

The main PI3-kinase isoform in cancer is the Class I PI3-kinase, p110 α (alpha) (U.S. Pat. No. 5,824,492; U.S. Pat. No. 5,846,824; U.S. Pat. No. 6,274,327). Other isoforms are implicated in cardiovascular and immune-inflammatory disease (Workman P (2004) Biochem Soc Trans 32:393-396; Patel et al (2004) Proceedings of the American Association of Cancer Research (Abstract LB-247) 95th Annual Meeting, March 27-31, Orlando, Fla., USA; Ahmadi K and Waterfield M D (2004) Encyclopedia of Biological Chemistry (Lennarz W J, Lane M D eds) Elsevier/Academic Press). The PI3 kinase/Akt/PTEN pathway is an attractive target for cancer drug development since such modulating or inhibitory agents would be expected to inhibit proliferation, reverse the repression of apoptosis and surmount resistance to cytotoxic agents in cancer cells (Folkes et al (2008) J. Med. Chem. 51:5522-5532; Yaguchi et al (2006) Jour. of the Nat. Cancer Inst. 98(8):545-556). The PI3K-PTEN-AKT signaling pathway is deregulated in a wide variety of cancers (Samuels Y, Wang Z, Bardellil A et al. High frequency of mutations of the PIK3CA gene in human cancers. (2004) Science; 304 (5670):554; Carpten J, Faber A L, Horn C. “A transforming mutation in the pleckstrin homology domain of AKT1 in cancer” (2007) Nature; 448:439-444).

GDC-0980 (Genentech, Inc., Roche, RG-7422) demonstrates broad activity in preclinical xenograft cancer models; breast, ovarian, lung, and prostate, and is being developed for the potential oral treatment of cancer including solid tumors and non-Hodgkin's lymphoma (Wagner A J; Burris III H A; de Bono J S et al AACR-NCI-EORTC International Congress (2009), 21st: November 17 (Abs B137) “Pharmacokinetics and Pharmacodynamic biomarkers for the dual PI3K/mTOR inhibitor GDC-0980: initial phase I evaluation”; U.S. Pat. No. 7,888,352; US 2009/0098135; US 2010/0233164). In March 2009, a phase I trial in patients with solid tumors or NHL was initiated; in April 2009, a second phase I trial began; these trials were ongoing in April 2010. In December 2010, a phase Ib combination trial in metastatic breast cancer was initiated. In July 2010, a phase II trial in metastatic breast cancer was planned for the first half of 2011; patients would receive GDC-0980 combined with hormonal therapy. Clinical results to date suggest that GDC-0980 may benefit patients with solid tumors or hematological malignancies. (Sutherlin D P, Belvin M, Bao L et al, American Association for Cancer Research Annual Meeting, (2011) 102nd: April 04 (Abs 2787)).

Mesothelioma, more precisely malignant mesothelioma, is a rare form of cancer that develops from the protective lining that covers many of the body's internal organs, the mesothelium (“Current concepts in malignant pleural mesothelioma”, Kaufman, Andrew J.; Pass, Harvey I, Expert Review of Anticancer Therapy (2008), 8(2), 293-303; “Malignant mesothelioma”, Pass, Harvey I.; Carbone, Michele; Chahinian, A. Philippe, Editor(s): Kufe, Donald W. Cancer Medicine 7 (2006), 1225-1236). Mesothelioma is usually caused by exposure to asbestos. Its most common site is the pleura, the outer lining of the lungs and internal chest wall. Mesothelioma may also occur in the peritoneum, the pericardium, or the tunica vaginalis (a sac that surrounds the testis). Most people who develop mesothelioma have worked on jobs where they inhaled asbestos and glass particles, or they have been exposed to asbestos dust and fiber in other ways. Unlike lung cancer, there is no association between mesothelioma and smoking, but smoking greatly increases the risk of other asbestos-induced cancers. Malignant pleural mesothelioma (MPM) is an aggressive tumor with poor prognosis, whose exposure to asbestos fibers is the main cause. The incidence of MPM is anticipated to increase worldwide during the first half of this century. MPM is notoriously refractory to most treatments (“Multidisciplinary treatment of malignant pleural mesothelioma” Ceresoli, Giovanni Luca; Gridelli, Cesare; Santoro, Armando, Oncologist (2007), 12(7), 850-863), and the only standard of care is cisplatin and antifolate first-line chemotherapy (“Targeted therapies in malignant pleural mesothelioma: a review of clinical studies”, Greillier, Laurent; Marco, Sabine; Barlesi, Fabrice. Anti-Cancer Drugs (2011), 22(3), 199-205; “Malignant pleural mesothelioma” Stahel, Rolf A.; Felley-Bosco, Emanuela; Opitz, Isabelle; Weder, Walter, Future Oncology (2009), 5(3), 391-402). Pemetrexed is currently approved in combination with cisplatin for first line treatment in patients with unresectable malignant pleural mesothelioma (“The role of pemetrexed in the pharmacotherapy of malignant pleural mesothelioma” Zucali, P. A., et al, Clinical Medicine Insights: Therapeutics (2010), 2 797-808; “Chemotherapy of malignant pleural mesothelioma: Where are we now and where are we going?” Hillerdal, Gunnar, Annals of Respiratory Medicine (2010), 1(2), 17-21). Chemotherapeutic and radiotherapeutic resistance makes malignant pleural mesothelioma (MPM) difficult to manage, even though encouraging results were achieved with multimodality treatment. Tyrosine kinase inhibitors (TKIs) targeting growth factors like vandetanib, dasatinib, and angiogenesis inhibitors like bevacizumab, are among the most promising agents under evaluation in clinical trials (“Molecular targets in malignant pleural mesothelioma treatment” Pasello, Giulia; Favaretto, Adolfo. U. O., Current Drug Targets (2009), 10(12), 1235-1244; “Malignant pleural mesothelioma”, Tsao, Anne S.; Wistuba, Ignacio; Roth, Jack A.; Kindler, Hedy Lee, Journal of Clinical Oncology (2009), 27(12), 2081-2090; “Malignant pleural mesothelioma: medical treatment update” Vorobiof, Daniel A.; Mafafo, Keorapetse, Clinical Lung Cancer (2009), 10(2), 112-117; “Chemotherapy of malignant pleural mesothelioma” Bertino, Pietro; Carbone, Michele; Pass, Harvey, Expert Opinion on Pharmacotherapy (2009), 10(1), 99-107; “Molecular targets and targeted therapies for malignant mesothelioma” Palumbo, Camilla; Bei, Roberto; Procopio, Antonio; Modesti, Andrea, Current Medicinal Chemistry (2008), 15(9), 855-867). Future directions for treatment include development of biomarkers for the potential screening of high risk, asbestos exposed individuals, and a greater understanding of the pathways involved in mesothelial carcinogenesis (“Therapeutic approaches to malignant mesothelioma” Pass, Harvey I.; Hahn, Stephen; Vogelzang, Nicholas, Editor(s): Craighead, John E.; Gibbs, Allen R. Asbestos and Its Diseases (2008) 326-345).

### SUMMARY OF THE INVENTION

The invention relates generally to methods of treating mesothelioma patients with the dual mTOR/PI3K inhibitor GDC-0980, named as (S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one, having the structure:

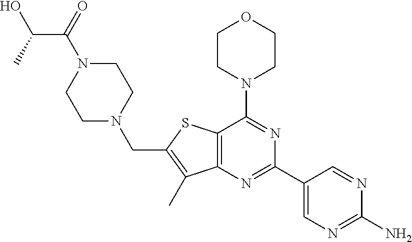

and stereoisomers, geometric isomers, tautomers, and pharmaceutically acceptable salts thereof.

The invention includes methods where the patient has malignant pleural mesothelioma and/or the patient has been previously treated with chemotherapy, radiotherapy, and/or surgical resection, including treatments with pemetrexed, bevacizumab, cisplatin, gemcitabine, vinorelbine, imatinib, dasatinib, erlotinib, sunitinib, or sorafenib.

An aspect of the invention includes administering GDC-0980 daily at three week or four week intervals to the patient, including where the three week interval is followed by a one week holiday interval where the patient is not administered GDC-0980.

An exemplary embodiment of the methods is where GDC-0980 is administered orally.

An exemplary embodiment of the methods is where the therapeutically effective amount of GDC-0980 is 1 mg to 100 mg per day of patient body weight, or 10 mg to 50 mg per day of patient body weight.

An exemplary embodiment of the methods is where the patient is also administered a chemotherapeutic agent selected from selected from pemetrexed, bevacizumab, cisplatin, gemcitabine, vinorelbine, imatinib, dasatinib, erlotinib, sunitinib, or sorafenib.

An exemplary embodiment of the methods is where GDC-0980 is formulated with an ingredient selected from microcrystalline cellulose, lactose monohydrate, croscarmellose sodium, copovidone, and magnesium stearate.

An exemplary embodiment of the methods is where GDC-0980 is formulated with a pharmaceutically acceptable glidant selected from silicon dioxide, powdered cellulose, microcrystalline cellulose, metallic stearates, sodium aluminosilicate, sodium benzoate, calcium carbonate, calcium silicate, corn starch, magnesium carbonate, asbestos free talc, stearowet C, starch, starch 1500, magnesium lauryl sulfate, magnesium oxide, and combinations thereof.

The invention relates generally to methods of treating mesothelioma patients, and to the use of for treating mesothelioma patients, with the dual mTOR/PI3K inhibitor GDC-0980 and stereoisomers, geometric isomers, tautomers, and pharmaceutically acceptable salts thereof.

An aspect of the invention includes GDC-0980, named as (S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one for use for treating malignant pleural mesothelioma and/or for treating patients where the patient has been previously treated with chemotherapy, radiotherapy, and/or surgical resection, including treatments with pemetrexed, bevacizumab, cisplatin, gemcitabine, vinorelbine, imatinib, dasatinib, erlotinib, sunitinib, or sorafenib.

An aspect of the invention includes the use of GDC-0980, named as (S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one for treating malignant pleural mesothelioma and/or for treating patients where the patient has been previously treated with chemotherapy, radiotherapy, and/or surgical resection, including treatments with pemetrexed, bevacizumab, cisplatin, gemcitabine, vinorelbine, imatinib, dasatinib, erlotinib, sunitinib, or sorafenib.

### DETAILED DESCRIPTION OF EXEMPLARY EMBODIMENTS

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents which may be included within the scope of the present invention as defined by the claims. One skilled in the art will recognize many methods and materials similar or equivalent to those described herein, which could be used in the practice of the present invention. The present invention is in no way limited to the methods and materials described. In the event that one or more of the incorporated literature, patents, and similar materials differs from or contradicts this application, including but not limited to defined terms, term usage, described techniques, or the like, this application controls.

### DEFINITIONS

The words “comprise,” “comprising,” “include,” “including,” and “includes” when used in this specification and claims are intended to specify the presence of stated features, integers, components, or steps, but they do not preclude the presence or addition of one or more other features, integers, components, steps, or groups thereof.

The terms “treat” and “treatment” refer to both therapeutic treatment and prophylactic or preventative measures, wherein the object is to prevent or slow down (lessen) an undesired physiological change or disorder, such as the growth, development or spread of a hyperproliferative condition, such as cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The terms “cancer” and “cancerous” refer to or describe the physiological condition in mammals that is typically characterized by unregulated cell growth. A “tumor” comprises one or more cancerous cells. Examples of cancer include, but are not limited to, mesothelioma, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

“Progression-Free Survival” (PFS) is the time from the first day of treatment to documented disease progression (including isolated CNS progression) or death from any cause on study, whichever occurs first.

“Overall Survival” is the time from first day of treatment to death from any cause.

A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy. Examples of chemotherapeutic agents include: pemetrexed (ALIMTA®, Eli Lilly Co., CAS No. 137281-23-3), erlotinib (TARCEVA®, Genentech/OSI Pharm., CAS No. 183321-74-6), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine,dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo[4.3.0]nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethyl-ethanamine, NOLVADEX®, ISTUBAL®, VALODEX®), and doxorubicin (ADRIAMYCIN®), Akti-1/2, HPPD, and rapamycin.

More examples of chemotherapeutic agents include: dasatinib (SPRYCEL®, BMS-354825, Bristol Myers Squibb, CAS Reg. No. 302962-49-8), oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib (GLEEVEC®, Novartis), XL-518 (MEK inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, Il), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, calicheamicin gamma1I, calicheamicin omegaI1 (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (T-2 toxin, verracurin A, roridin A and anguidine); urethan; vindesine; dacarbazine; mannomustine; mitobronitol; mitolactol; pipobroman; gacytosine; arabinoside (Ara-C); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®, CAS Reg. No. 71486-22-1); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and derivatives of any of the above.

The term “package insert” is used to refer to instructions customarily included in commercial packages of therapeutic products, that contain information about the indications, usage, dosage, administration, contraindications and/or warnings concerning the use of such therapeutic products.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

An “adverse event” (AE) is any unfavorable and unintended sign, symptom, or disease temporally associated with the use of an investigational (medicinal) product or other protocol-imposed intervention, regardless of attribution; and includes: AEs not previously observed in the patient that emerge during the protocol-specified AE reporting period, including signs or symptoms associated with breast cancer that were not present before the AE reporting period; complications that occur as a result of protocol-mandated interventions (e.g., invasive procedures such as biopsies); if applicable, AE that occur before assignment of study treatment associated with medication washout, no treatment run-in, or other protocol-mandated intervention; Preexisting medical conditions (other than the condition being studied) judged by the investigator to have worsened in severity or frequency or changed in character during the protocol-specified AE reporting period.

An adverse event is classified as a “Serious Adverse Events” (SAE) if it meets the following criteria: results in death (i.e., the AE actually causes or leads to death); life threatening (i.e., the AE, in the view of the investigator, places the patient at immediate risk of death, but not including an AE that, had it occurred in a more severe form, might have caused death); requires or prolongs inpatient hospitalization; results in persistent or significant disability/incapacity (i.e., the AE results in substantial disruption of the patient's ability to conduct normal life functions); results in a congenital anomaly/birth defect in a neonate/infant born to a mother exposed to the investigational product; or is considered a significant medical event by the investigator based on medical judgment (e.g., may jeopardize the patient or may require medical/surgical intervention to prevent one of the outcomes listed above). All AEs that do not meet any of the criteria for serious are regarded as non-serious AEs. The terms “severe” and “serious” are not synonymous. Severity (or intensity) refers to the grade of a specific AE, e.g., mild (Grade 1), moderate (Grade 2), or severe (Grade 3) myocardial infarction (see Section 5.2.2). “Serious” is a regulatory definition (see previous definition) and is based on patient or event outcome or action criteria usually associated with events that pose a threat to a patient's life or functioning. Seriousness (not severity) serves as the guide for defining regulatory reporting obligations from the Sponsor to applicable regulatory authorities. Severity and seriousness should be independently assessed when recording AEs and SAEs on the eCRF.

### ABBREVIATIONS AND DEFINITION OF TERMS

| | |
|---|---|
| AE | adverse event |
| CBR | clinical benefit rate |
| CHF | congestive heart failure |
| CL | clearance |
| CNS | central nervous system |
| CT | computed tomography |
| DLT | dose-limiting toxicity |
| ECG | electrocardiogram |
| ECHO | echocardiogram |
| eCRF | electronic Case Report Form |
| EDC | electronic data capture |
| FACT-B | Functional Assessment of Cancer Therapy-Breast |
| FDA | Food and Drug Administration |
| FISH | fluorescence in situ hybridization |
| HER2 | human epidermal growth factor receptor 2 |
| IHC | immunohistochemistry |
| IND | Investigational New Drug |
| IRB | institutional review board |
| IRF | independent review facility |
| LS | liquid chromatography |
| MS | mass spectrometry |
| LVEF | left ventricular ejection fraction |
| MRI | magnetic resonance imaging |
| MTD | maximum tolerated dose |
| NCI CTCAE | National Cancer Institute Common Terminology |
| | Criteria for Adverse Events |
| PFS | progression-free survival |
| PK | pharmacokinetic |
| PR | partial response |
| RECIST | Response Evaluation Criteria in Solid Tumors |
| RR | response rate |
| RT-PCR | reverse transcriptase polymerase chain reaction |
| SAE | serious adverse event |
| SD | stable disease |
| SWFI | Sterile Water for Injection |
| ULN | upper limit of normal |

### GDC-0980

The present invention includes therapeutic treatments with GDC-0980, a small molecule inhibitor of PI3K and mTOR, (CAS Reg. No. 1032754-93-0), which has the structure:

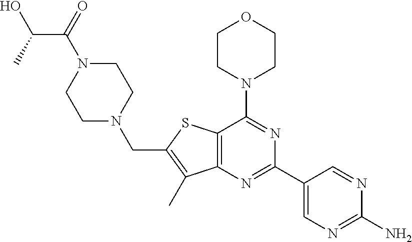

and may be named: (S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one (U.S. Pat. No. 7,888,352; US 2009/0098135; US 2010/0233164). As used herein, GDC-0980 includes all stereoisomers, geometric isomers, tautomers, and pharmaceutically acceptable salts thereof.

GDC-0980 is a potent, selective, oral inhibitor of Class I PI3K and mTOR kinase with the following in vitro biochemical IC50s against Class I isoforms of PI3K: p110a (alpha) 4.8 nM; p110β (beta) 26.8 nM; p110 (gamma) 13.8 nM; p110d (delta) 6.7 nM; mTOR Ki 17.3 nM. GDC-0980 was selective for PI3K versus a large panel of kinases (>145), including other members of the phosphatidylinositol kinase family. In PC3 and MCF7-neo/HER2 cell lines, the compound demonstrated IC50 values of 307 and 320 nM, respectively. GDC-0980 was stable in human microsomes and hepatocytes, exhibited low activity against hERG (IC50 >100 microM) and did not elicit significant responses in a receptor screening assay (n=68; GDC-0980=10 microM). Moderate-to-high clearance was observed in rodents (60 ml/min/kg) and dogs (12 ml/min/kg). The terminal half-life of the compound was 6 to 18 h, with dose-proportional increases in AUC and Cmax values following a single oral dose. GDC-0980 (25 to 150 mg/kg qd po) was efficacious across multiple xenograft models, including mouse PC3 PTEN-prostate and MCF7.1 E545K breast xenograft models. In a MDA-MB-361.1 breast cancer xenograft model, GDC-0980 produced significant growth inhibition at a minimum dose of 1.0 mg/kg QD.

### Clinical Study Objectives

This Phase I study evaluated the safety and efficacy (as evidenced by objective response rate and duration of objective response) of GDC-0980 in patients with prior treatments, as described below.

The primary objectives of the Phase I clinical study were to assess the objective response rate (through independent radiologic review) of patients treated with GDC-0980 and to characterize the safety and tolerability of GDC-0980 in this patient population. The secondary objectives of this study were to further characterize the efficacy of GDC-0980 in this patient population, as measured by duration of objective response, clinical benefit rate (CBR), which is the proportion of patients with CR, PR, and SD at 6 months, overall survival, and progression-free survival (PFS) end points based on independent radiologic review, and to characterize the pharmacokinetics of GDC-0980 in this patient population.

The exploratory objectives of this study were to: (i) investigate whether the level of PIK3CA gene amplification (as assessed by fluorescence in situ hybridization “FISH”) and/or mRNA expression, as assessed by reverse transcriptase polymerase chain reaction (RT-PCR) in archival tumor tissue correlates with GDC-0980 efficacy; (ii) to investigate whether levels of expression of PI3KCA in archival tumor tissue correlate with GDC-0980 efficacy; (iii) conduct an exploratory exposure—effect analysis to investigate the relationship between the pharmacokinetics of GDC-0980 and drug effect (e.g., efficacy, safety); and (iv) measure change from baseline in pAKT and/or other pathway biomarkers in platelet-rich plasma and in malignant plasma cells (multiple myeloma patients).

A nonclinical PK/PD model of tumor growth inhibition was developed using the indirect response model (Jusko W J. “Pharmacodynamics of chemotherapeutic effects: dose-time-response relationships for phase-nonspecific agents”, J Pharm Sci (1971) 60:892-5) to fit exposure and growth inhibition data based on four sets of experiments examining varying schedules and doses in the MDA-MB-361.1 xenografts. The PK/PD model was then used to predict minimal exposure needed to achieve 50% tumor growth inhibition, using the predicted human PK data and the nonclinical PD data. This exposure was preliminarily determined to be an AUC of approximately 1.7 μM·hr. On the basis of MLP allometric scaling, a daily dose of approximately 18 mg may achieve this exposure. Dose escalation will continue until the MTD, even if 1.7 μM·hr exposure is surpassed and well tolerated, since it is well established that tumor xenograft models are not absolutely predictive of clinical response (Sausville and Burger 2006).

Efficacy Assessments: Tumor responses are categorized as complete response (CR), partial response (PR), stable disease (SD), or progressive disease (PD) according to the Response Evaluation Criteria for Solid Tumors (RECIST) according to Therasse P, Arbuck S G, Eisenhauser E A, Wanders J, Kaplan R S, Rubinstein L, et al. “New guidelines to evaluate the response to treatment in solid tumors”, J Natl Cancer Inst (2000) 92:205-16. Tumor assessments (CT and/or magnetic resonance imaging [MRI] scans) are performed approximately every 6 weeks irrespective of dose delays, interruptions, or reductions. Bone and brain scans (either CT or MRI) may be performed at baseline and if clinically indicated during the study. Patient management decisions were made based on tumor assessments performed by investigators. The primary study endpoints related to response were determined by an independent radiologic review of patient scans, with assessments based on investigator reporting being secondary. If each non-target lesion cannot be assessed at a follow-up tumor assessment timepoint, patients may still be considered evaluable for timepoint response as long as all target lesions are measured. Patients with non-target lesions that were not assessed at a timepoint were assessed as having a partial response, stable disease, or progressive disease. All lesions, target and non-target, were assessed before a response was considered confirmed. To reduce the frequency of unevaluable non-target lesions, bone lesions visible on baseline CT scan as non-target lesion(s) for follow-up, were compared with bone lesions identified on the baseline bone scan where possible if lesions are not visible on both modalities or as easily and reproducibly assessed.

### Methods of Treatment: Rationale for Evaluation of a Cohort of Refractory Pleural Mesothelioma Patients in Stage 2

Patients with relapsed or refractory pleural mesothelioma were enrolled in the expansion stage (Stage 2) to characterize the safety, PK, and PD profiles and preliminary effect of GDC-0980 at the proposed single-agent dose and schedule for future studies. PI3-kinase and mTOR are attractive therapeutic targets in malignant pleural mesothelioma. Mesothelioma cell lines have higher levels of activated Ras when compared to nontransformed mesothelial cell lines (Patel M R, Jacobson B A, De A, et al. “Ras pathway activation in malignant mesothelioma” J Thorac Oncol (2007) 9:789-95). While Ras mutations rarely occur in mesothelioma, there is evidence that Ras and PI3K/AKT/mTOR activation may occur through diverse cellular mechanisms. Receptor tyrosine kinases (RTKs) such as epidermal growth factor receptor, insulin-like growth factor receptor, and c-MET that signal through Ras and PI3K/Akt/mTOR, have been shown to be activated in mesotheliomas (Tolnay E, Kuhnen C Wiethege T, et al. “Hepatocyte growth factor/scatter factor and its receptor c-Met are overexpressed and associated with an increased microvessel density in malignant pleural mesothelioma” J Cancer Res Clin Oncol (1998) 124:291-96; Thirkettle I, Harvey P, Hasleton P S, et al. “Immunoreactivity for cadherins, HGF/SF, met and erbB-2 in pleural malignant mesotheliomas” Histopathology (2000) 36:522-528). An additional molecular mechanism of PI3K/mTOR signaling deregulation is inactivating mutations in the tumor suppressor gene, PTEN. Loss of PTEN expression measured by immunohistochemistry was documented in 62% of 341 of human mesothelioma samples and was negatively correlated with overall survival independently of histologic subtype (Optiz et al. 2008). Biallelic inactivation of the NF2 tumor suppressor gene which encodes the protein Merlin has been identified in approximately 40-50% of malignant mesotheliomas (Bianchi A B, Mitsunaga S I, Cheng J Q, et al. “High frequency of inactivating mutations in the neurofibromatosis type 2 gene (NF2) in primary malignant mesotheliomas”. Proc Nat Acad Sci USA (1995) 92:10854-8; Sekido Y, Pass H I, Bader S, et al. “Neurofibromatosis type 2 (NF2) gene is somatically mutated in mesothelioma but not in lung cancer”. Cancer Res (1995) 55:1227-31). Murine models of heterozygous deletion of Nf2 have elevated levels of phosphorylated AKT (Altomare D A, Huihong Y, Guang-Hui X, et al. “Human and Mouse mesotheliomas exhibit elevated AKT/PKB activity, which can be targeted pharmacologically to inhibit tumor cell growth”, Oncogene (2005) 24:6080-9). Recently, loss of Merlin expression has been shown to result in activation of mTOR signaling in malignant mesothelioma samples (Lopez-Lago M A, Okada T, Murillo M M, et al. “Loss of the tumor suppressor NF2/Merlin constitutively activates integrin-dependent mTORC1 signaling”, Mol Cell Biol (2009) 15:4235-49).

Consistent with the frequency of RTK, PTEN and NF2 alterations in mesothelioma and their predicted biochemical alterations in PI3K/AKT/mTOR signaling, 60% of human mesothelioma samples demonstrated elevated phosphorylation of AKT and mTOR. Moreover, pharmacologic inhibition of PI3K affected cell survival and when combined with cisplatin resulted in enhanced response over either agent alone (Altomare D A, (2005). Additionally mesothelioma cells lack of Merlin expression was correlated with activated mTOR signaling and increased sensitivity to rapamycin (Lopez-Lago M A, (2009). These data combined with the clinical activity described during dose escalation in this Phase I study justify further exploration of the safety and efficacy of GDC-0980 in refractory pleural mesothelioma.

Primary outcome measures include: (i) occurrence of Dose Limiting Toxicities (DLT), as defined by NCI CTCAE grade and associated dose of GDC-0980; (ii) occurrence of adverse events by NCI CTCAE grade and associated dose of GDC-0980; (iii) occurrence of Grade 3 and 4 abnormalities in safety-related laboratory parameters and associated dose of GDC-0980; (iv) PK parameters, including time to maximum concentration (tmax), Cmax, minimum concentration (Cmin), t1/2; and (v) exposure (AUC) after single and multiple doses of GDC-0980.

Secondary outcome measures include: (i) Cmax and AUC under fasting conditions; and (ii) best overall response, duration of objective response (OR), and progression-free survival (PFS) for patients with measurable disease according to RECIST, for patients with NHL according to IWG, for patients with MM (multiple myeloma) according to EBMT, and for patients with mesothelioma in Stage 2 by modified RECIST.

### Antitumor Activity

Patients treated by the methods of the invention include those with diagnosed mesothelioma and a prior treatment history including chemotherapy, radiotherapy, and/or surgical resection.

Primary analysis of the antitumor activity data included patient assessments:

A 55-year-old woman, diagnosed with adrenal cortical cancer in 2004, was previously treated with surgery for extensive metastatic disease in the liver, pelvic wall, and peritoneum. The patient enrolled at 40 mg QD GDC-0980 (AUC 0-24 h ˜4.1 μM·hr) on a 28/28 day schedule and demonstrated a 22% decrease in target lesions by RECIST at the end of Cycle 1 and 39% decrease after 3 weeks of treatment in Cycle 2. GDC-0980 was interrupted in Cycle 2 due to adverse events of Grade 2 increased ALT and Grade 3 rash. The patient remained on study at a reduced dose of GDC-0980. The right subhepatic lesion is shown in PET images.

A 60-year-old man, diagnosed with epitheloid mesothelioma cancer four years earlier, was previously treated with XRT and cisplatin/pemetrexed. Analysis of archival tumor tissue showed a R88Q mutation in PIK3CA exon 2. The patient enrolled at 8 mg QD GDC-0980 (AUC0-24 h ˜0.4 μM·hr) on a 21/28 day schedule and demonstrated a 26% decrease in target lesions by RECIST at the end of Cycle 2. The patient was on study for about 4.5 months before progression of disease. The mediastinal disease is shown in PET images.

A 32-year-old woman, diagnosed with peritoneal mesothelioma cancer seven years earlier, was previously treated with XRT and cisplatin/pemetrexed. The patient enrolled at 32 mg QD GDC-0980 (AUC0-24 h ˜4.4 μM·hr) on a 21/28 day schedule and demonstrated a 28% decrease in target lesions by RECIST at the end of Cycle 6. The patient is currently on study after 1 year 3 months. A peritoneal nodule in the left iliac fossa is shown in PET images.

A 73 year-old man, with epitheloid pleural mesothelioma diagnosed 3 years earlier was previously treated with both cisplatinum/pemetrexed and carboplatinum/pemetrexed regimens. The patient enrolled at the 50 mg QD dose of GDC-0980 (AUC0-24 h ˜3 μM·hr) on a 21/28 day schedule and demonstrated a 26% decrease in target lesions by RECIST at the end of Cycles 1 and 2.

A 72 year-old woman, with pleural mesothelioma diagnosed 3 years earlier was previously treated with both cisplatin/pemetrexed and carboplatin/pemetrexed regimens. The patient enrolled at the 40 mg QD dose of GDC-0980 on a 28/28 day schedule and demonstrated a decrease in target lesions by RECIST at the end of Cycle 1 (amount of decrease preliminary and being quantitated).

A 59 year-old man, with pleural mesothelioma diagnosed 2 years earlier was previously treated with cisplatin/pemetrexed. The patient enrolled at the 50 mg QD dose of GDC-0980 (AUC0-24 h ˜3 μM·hr) on a 21/28 day schedule and demonstrated a decrease in target lesions by RECIST at the end of Cycles 1 (amount of decrease preliminary and being quantitated).

### Formulations

GDC-0980 may be formulated in accordance with standard pharmaceutical practice for use in a therapeutic combination for therapeutic treatment (including prophylactic treatment) of hyperproliferative disorders in mammals including humans. The invention provides a pharmaceutical composition comprising GDC-0980 in association with one or more pharmaceutically acceptable carrier, glidant, diluent, or excipient.

Suitable carriers, diluents, glidants, and excipients are well known to those skilled in the art and include materials such as carbohydrates, waxes, water soluble and/or swellable polymers, hydrophilic or hydrophobic materials, gelatin, oils, solvents, water and the like.

The formulations may be prepared using conventional dissolution and mixing procedures. The compound of the present invention is typically formulated into pharmaceutical dosage forms to provide an easily controllable dosage of the drug and to enable patient compliance with the prescribed regimen.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Pharmaceutical formulations of the compounds of the present invention may be prepared for various routes and types of administration with pharmaceutically acceptable diluents, carriers, excipients, glidants or stabilizers (Remington's Pharmaceutical Sciences (1995) 18th edition, Mack Publ. Co., Easton, Pa.), in the form of a lyophilized formulation, milled powder, or an aqueous solution. Formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8.

The pharmaceutical formulation is preferably sterile. In particular, formulations to be used for in vivo administration must be sterile. Such sterilization is readily accomplished by filtration through sterile filtration membranes.

The pharmaceutical formulation ordinarily can be stored as a solid composition, a tablet, a pill, a capsule, a lyophilized formulation or as an aqueous solution.

The pharmaceutical formulations of the invention will be dosed and administered in a fashion, i.e., amounts, concentrations, schedules, course, vehicles and route of administration, consistent with good medical practice. Factors for consideration in this context include the particular disorder being treated, the clinical condition of the individual patient, the cause of the disorder, the site of delivery of the agent, the method of administration, the scheduling of administration, and other factors known to medical practitioners.

As a general proposition, the initial pharmaceutically effective amount of GDC-0980 administered per dose will be in the range of about 1 to 100 mg per day (QD) of patient body weight. The administered dose may be about 10 to about 50 mg per day of patient body weight. In particular, the daily dose may be 20, 30 mg or 40 mg.

Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl, ethanol, or benzylalcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, including Tween 80, PLURONICS™ or polyethylene glycol (PEG), including PEG400. The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition, (1995) Mack Publ. Co., Easton, Pa. Other examples of drug formulations can be found in Liberman, H. A. and Lachman, L., Eds., Pharmaceutical Dosage Forms, Marcel Decker, Vol 3, 2^{nd} Ed., New York, N.Y.

Pharmaceutically acceptable glidants may be selected from silicon dioxide, powdered cellulose, microcrystalline cellulose, metallic stearates, sodium aluminosilicate, sodium benzoate, calcium carbonate, calcium silicate, corn starch, magnesium carbonate, asbestos free talc, stearowet C, starch, starch 1500, magnesium lauryl sulfate, magnesium oxide, and combinations thereof.

The pharmaceutical formulations include those suitable for the administration routes detailed herein. The formulations may conveniently be presented in unit dosage form and may be prepared by any of the methods well known in the art of pharmacy. Techniques and formulations generally are found in Remington's Pharmaceutical Sciences 18^{th} Ed. (1995) Mack Publishing Co., Easton, Pa. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Pharmaceutical compositions may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may be a solution or a suspension in a non-toxic parenterally acceptable diluent or solvent, such as a solution in 1,3-butanediol or prepared from a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of GDC-0980 with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

### Combination Therapy

GDC-0980 may be employed in combination with other chemotherapeutic agents for the treatment of a hyperproliferative disease or disorder, including tumors, cancers, and neoplastic tissue, along with pre-malignant and non-neoplastic or non-malignant hyperproliferative disorders. In certain embodiments, GDC-0980 is combined in a pharmaceutical combination formulation, or dosing regimen as combination therapy, with a second compound that has anti-hyperproliferative properties or that is useful for treating the hyperproliferative disorder. The second compound of the pharmaceutical combination formulation or dosing regimen preferably has complementary activities to GDC-0980, and such that they do not adversely affect each other. Such compounds are suitably present in combination in amounts that are effective for the purpose intended. In one embodiment, a composition of this invention comprises GDC-0980 in combination with a chemotherapeutic agent such as described herein.

Therapeutic combinations of the invention include a formulation, dosing regimen, or other course of treatment comprising the administration of GDC-0980, and a chemotherapeutic agent selected from pemetrexed, bevacizumab, cisplatin, gemcitabine, vinorelbine, imatinib, dasatinib, erlotinib, sunitinib, or sorafenib. Other chemotherapeutic agents which can be combined in a course of therapy with GDC-0980 include docetaxel, 5-FU, PD-0325901, carboplatin, paclitaxel, trastuzumab, pertuzumab, temozolomide, tamoxifen, doxorubicin, Akti-1/2, HPPD, rapamycin, and lapatinib (US 2010/0098135), dexamethasone, thioTEPA, doxorubicin, vincristine, rituximab, cyclophosphamide, prednisone, melphalan, lenalidomide, bortezomib, rapamycin, and cytarabine (US 2010/0233164), for separate, simultaneous or sequential use in the treatment of mesothelioma, and other hyperproliferative disorders.

The combination therapy may be administered as a simultaneous or sequential regimen. When administered sequentially, the combination may be administered in two or more administrations. The combined administration includes coadministration, using separate formulations or a single pharmaceutical formulation, and consecutive administration in either order, wherein preferably there is a time period while both (or all) active agents simultaneously exert their biological activities.

Suitable dosages for any of the above coadministered agents are those presently used and may be lowered due to the combined action (synergy) of the newly identified agent and other chemotherapeutic agents or treatments.

In a particular embodiment of anti-cancer therapy, GDC-0980 may be combined with a chemotherapeutic agent, including hormonal or antibody agents such as those described herein, as well as combined with surgical therapy and radiotherapy. The amounts of GDC-0980 and the other pharmaceutically active chemotherapeutic agent(s) and the relative timings of administration will be selected in order to achieve the desired combined therapeutic effect.

### Administration of GDC-0980

Pharmaceutical compositions of GDC-0980 may be administered by any route appropriate to the condition to be treated. Suitable routes include oral, parenteral (including subcutaneous, intramuscular, intravenous, intraarterial, inhalation, intradermal, intrathecal, epidural, and infusion techniques), transdermal, rectal, nasal, topical (including buccal and sublingual), vaginal, intraperitoneal, intrapulmonary and intranasal. Topical administration can also involve the use of transdermal administration such as transdermal patches or iontophoresis devices. Where the compound is administered orally, it may be formulated as a pill, capsule, tablet, etc. with a pharmaceutically acceptable carrier, glidant, or excipient. Where the compound is administered parenterally, it may be formulated with a pharmaceutically acceptable parenteral vehicle or diluent, and in a unit dosage injectable form, as detailed below.

A dose of GDC-0980 to treat human patients may range from about 1 mg to about 100 mg. A daily dose may be 10 mg, 20 mg, 30 mg, 40 mg, or 50 mg. The dose of GDC-0980 may be administered once every six weeks, once every three or four weeks, weekly, daily, or more frequently, depending on the pharmacokinetic (PK) and pharmacodynamic (PD) properties, including absorption, distribution, metabolism, and excretion. The dosing schedule may comprise an interval of treatment with daily or twice daily doses of GDC-0980 for about 3 weeks, then a dosing holiday of about one week. The dosing schedule may be continuous dosing for a period such as 4 weeks of daily or twice daily doses of GDC-0980. The dosing schedule may be followed by more intervals of dosing/holiday depending on disease progression and tolerance.

A dose of the chemotherapeutic agent, if used in combination with GDC-0980, may range from about 1 mg to about 1000 mg. The chemotherapeutic agent may be administered once every six weeks, once every three weeks, weekly, or more frequently, such as once or twice per day. In addition, toxicity factors may influence the dosage and administration regimen. When administered orally, the pill, capsule, or tablet may be ingested daily or less frequently for a specified period of time. The regimen may be repeated for a number of cycles of therapy.

### Articles of Manufacture

In another embodiment of the invention, an article of manufacture, or “kit”, containing GDC-0980 useful for the treatment of the diseases and disorders described above is provided. In one embodiment, the kit comprises a container comprising GDC-0980. The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, for example, bottles, vials, syringes, blister pack, etc. The container may be formed from a variety of materials such as glass or plastic. The container may hold GDC-0980 or a formulation thereof which is effective for treating the condition and may have a sterile access port (for example, the container may be an intravenous solution bag or a vial having a stopper pierceable by a hypodermic injection needle). At least one active agent in the composition is GDC-0980, which may be in lyophilized form. The label or package insert indicates that the composition is used for treating the condition of choice, such as cancer. In one embodiment, the label or package inserts indicates that the composition comprising GDC-0980 can be used to treat mesothelioma. The label or package insert may also indicate that the composition can be used to treat other disorders. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of GDC-0980 and, if present, the second pharmaceutical formulation. For example, if the kit comprises a first composition comprising GDC-0980 and a second pharmaceutical formulation, the kit may further comprise directions for the simultaneous, sequential or separate administration of the first and second pharmaceutical compositions to a patient in need thereof.

In another embodiment, the kits are suitable for the delivery of solid oral forms of GDC-0980, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

According to one embodiment, a kit may comprise (a) a first container with GDC-0980 contained therein; and optionally (b) a second container with a second pharmaceutical formulation contained therein, wherein the second pharmaceutical formulation comprises a second compound with anti-hyperproliferative activity. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Where the kit comprises a composition of GDC-0980 and a second therapeutic agent, i.e. the chemotherapeutic agent, the kit may comprise a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

### EXAMPLES

In order to illustrate the invention, the following examples are included. However, it is to be understood that these examples do not limit the invention and are only meant to suggest a method of practicing the invention.

### Example 1

### Dosage, Formulation, Administration, and Storage of GDC-0980

GDC-0980 was prepared and formulated according to the procedures in Example 201 of U.S. Pat. No. 7,888,352, and WO 2009/055730.

A powder-in-capsule formulation was used in the Phase I clinical studies of GDC-0980. The drug product is a powder-in-capsule formulation consisting of API as powder, free base GDC-0980 in hard gelatin capsule shells. The administered drug product was available in capsules of three strengths: 1, 5, 15, and 25 mg (active). The 1-mg capsules are Size 3 and opaque Swedish orange. The 5-mg capsules are Size 2 and opaque dark green. The 15-mg capsules are Size 1 and opaque white. The 25-mg capsules are Size 0 with an opaque white body and an opaque dark green cap. The only excipient in the GDC-0980 drug product is the hard gelatin capsule shell.

A film-coated tablet for oral administration of GDC-0980 is prepared for a Phase II study. The composition of a 10 mg GDC-0980 tablet is described in detail in Table 1. Similar tablets at other doses, including 30, 50, and 100 mg, of GDC-0980 may be prepared with the same or similar composition at proportional amounts of Ingredients and Film Coat, and analyzed by the same tests and assays.

TABLE 1
GDC-0980 10 mg tablet composition

| | | Nominal | % |
|---|---|---|---|
| | | Amount | (w/w) |
| | | (mg) per | of |
| Ingredient | Function | Tablet | Blend |
| Tablet Core | | | |
| GDC-0980 | Active Ingredient | 10.0^{a} | 10.0 |
| | (API) | | |
| Microcrystalline Cellulose | Filler | 40.8^{b} | 40.8 |
| Lactose Monohydrate | Filler | 42.2 | 42.2 |
| Croscarmellose Sodium | Super-Disintegrant | 3.0 | 3.0 |
| Copovidone | Binder | 3.0 | 3.0 |
| Magnesium Stearate | Lubricant | 1.0 | 1.0 |
| (Non-Bovine) | | | |
| Total Weight (mg) | — | 100.0 | — |
| Film Coat | | | |
| Opadry II White^{d} | Color Coat | 5.0 | 5.0 |
| Purified Water^{c} | Solvent | NA | NA |
| Total Weight (mg) | — | 5.0 | |

NA—not applicable.
^{a}The amount of GDC-0980 is adjusted according to the potency of API.
^{b}The amount of microcrystalline cellulose is adjusted based on the actual amount of GDC-0980.
^{c}Removed during process.
^{d}Opadry II, White consists of polyethylene glycol, polyvinyl alcohol, titanium dioxide, and talc.

The 10 mg tablet formulation employs a dry granulation-roller compaction process to obtain free-flowing material, which is compressed into tablets. The compressed tablets are then coated into the final product, a film-coated tablet. The tablet formulation contains the following excipients: microcrystalline cellulose (filler), lactose monohydrate (filler), copovidone (binder), croscarmellose sodium (super-disintegrant), and magnesium stearate (lubricant). This dose was manufactured from a blend containing a 10% drug load (free-base equivalent of GDC-0980). The design of experiment (DOE) study had a half factorial with three center points design for the formulation changes and a full factorial design for the process changes. The combination of lactose and microcrystalline cellulose as fillers allowed greater processing flexibility because of the two fillers' different deformation mechanisms. Croscarmellose sodium (super-disintegrant), copovidone (binder), and magnesium stearate (lubricant) were selected as they are commonly used excipients for dry granulation. Dry granulation by roller compaction was used to increase the density of both formulation blends, improve the flow properties, and improve content uniformity of the tablets.

The round, white, film-coated tablet containing GDC-0980 was analyzed by various tests and assays, including: visual appearance, identity (HPLC, 1H NMR, UV) against reference standard, purity, water content, assay by HPLC for related substances, uniformity of dosage units, dissolution, storage stability (controlled temperature and relative humidity), and safety (microbial limit test).

For Cycle 1 of the Phase I study, a single dose of GDC-0980 is administered to patients on Day 1 in a clinical setting that can accommodate frequent blood draws over a period of up to 48 hours after the dose is administered. Daily dosing will begin on Day 8 and continue for 21 days (Days 8-28), followed by a 7-day off-drug observation period (Days 29-35). Subsequent cycles will be 28 days in length (21 days of daily dosing followed by a 7-day observation period). Additional cohorts may be added to examine the tolerability of a 28-day dosing schedule with no observation period after the MTD is exceeded defined for the 21-day dosing schedule. GDC-0980 should be taken on an empty stomach (fasting) in the morning, unless otherwise instructed. For all patients Days 1 and 15 (PK sampling days), patients should be fasted overnight (at least 8-10 hours) prior to their dose on those days. On other days, patients should avoid consuming anything but water for 1 hour before and 2 hours after GDC-0980 administration. Each dose should be taken with a minimum of 3-4 ounces of water. Patients should be instructed to take their GDC-0980 dose at least 2 hours prior to their first meal of the day and at approximately the same clock time (no earlier than 1 hour and no later than 4 hours after the scheduled time) each day, unless otherwise instructed such as for a clinic visit with PK sampling, tumor biopsy, or imaging study.

### Example 2

### Clinical Study of GDC-0980 in Treatment of Patients with Solid Tumors or Non-Hodgkins Lymphoma

Study Design

This is an open-label, multicenter, Phase I study using a 3+3 design to evaluate the safety, tolerability, and pharmacokinetics of escalating oral (po) doses of GDC-0980 administered to QD (once a day). This study will include patients with incurable, locally advanced or metastatic solid malignancy, or NHL that has progressed or failed to respond to at least one prior regimen or for which there is no standard therapy either does not exist or has proven ineffective or intolerable. Eligible patients had solid tumors refractory to standard treatments, ECOG performance status 0-1, life expectancy=12 weeks, HbA1c=1×ULN, fasting serum glucose=120 mg/dL. Patients with a history of Type 1 or 2 diabetes mellitus requiring regular medication were excluded. GDC-0980 was administered on Day 1, followed by 1 week of washout to evaluate single-dose PK and PD markers. GDC-0980 was then dosed QD for either 21 days or 28 days every 28-day cycle. Tumor assessments were performed after Cycles 1, 2, and then every 2 cycles using RECIST guidelines. Optional FDG-PET was obtained between Days 22-28 and Days 50-57.

The treatment period consists of two stages: Stage 1 (dose escalation) and Stage 2 (expansion). Stage 1 will examine the safety and pharmacokinetics of increasing doses of GDC-0980 administered once daily for 21 or 28 days of a 28-day cycle (see Stage 1 below). Stage 2 will enroll an additional 6 to 12-15 patients at the MTD and selected schedule to further characterize the safety, tolerability, and PK variability at the proposed dose and schedule for future studies. Stage 2 will have 3 additional patient cohorts that will each enroll up to 12 patients with one of the following tumor types: MM advanced breast cancer with a PIK3CA mutation, and relapsed or refractory pleural mesothelioma. Cycle 1 of either stage will be 35 days in length and will include a 7-day single-dose PK evaluation followed by continuous daily dosing for either 21 days followed by 7-day off-drug observation period, or 28 days with no off-drug observation period. Subsequent cycles will be 28 days (daily dosing for 21 days followed by 7 days off drug or daily dosing for 28 days).

In Stage 2, FDG-PET imaging will be mandatory for all solid tumor patients unless their initial FDG-PET scans showed no tumor uptake of FDG or they have a measurable lesion of appropriate size to be eligible for DCE-MRI. Tumor biopsies will continue to be optional in Cycle 1 of Stage 2 with the exception of patients with multiple myeloma (MM). FDG-PET results will not be used to make assessments of response or progression for decisions regarding continuation of study treatment or discontinuation of a patient, as FDG-PET has not been validated as an indicator of early response or progression in this setting. MM patients underwent bone marrow aspirate and trephine biopsy (BMA/biopsy) at baseline and once during Cycle 1.

Disease status will be assessed using the Response Evaluation Criteria in Solid Tumors (RECIST), or the International Working Group (IWG) response criteria for NHL. For Stage 2 expansion cohorts in MM the European Group for Blood and Bone Marrow Transplant (EBMT) response criteria for MM will be utilized. For mesothelioma patients in the expansion cohort, modified RECIST will be used for disease assessments (Example. Patients will undergo disease assessments close to or on Day 36 (i.e., after the first cycle) and after every even-numbered cycle (i.e., every two cycles), or earlier if clinically indicated. According to RECIST, tumor status for patients with solid tumors, and modified RECIST for pleural mesothelioma, will be categorized as a complete response, partial response, stable disease, or progressive disease, with objective response confirmed by repeat physical examination or image-based evaluation ≧4 weeks after the initial documentation. NHL or MM disease status will be assessed as described. Dosing beginning on Day 36 will be at the discretion of the investigator, after a careful assessment and thorough discussion of the potential risks and benefits of continued treatment with the patient. GDC-0980 administration will be discontinued in patients who: (1) experience a DLT during the DLT Assessment Window (Days 1-35 of the first cycle); (2) at any time during the study, experience disease progression or unacceptable toxicity, or (3) in their opinion or the opinion of the investigator, are not benefiting from GDC-0980.

An Early Termination Visit will be performed within 30 days after early treatment discontinuation; assessments will be the same as for the last scheduled treatment visit. An End of Study Visit will be performed 30 days after the last dose of GDC-0980 for patients with unresolved adverse events or abnormal laboratory values thought to be related to GDC-0980.

Study Objectives

Primary/Secondary Objectives were to: (1) Evaluate safety and tolerability and establish the maximum tolerated dose (MTD) of increasing oral doses of GDC-0980 on a 3-weeks-on/1-week-off (21/28) schedule and continuous (28/28) daily dosing schedule; (2) Characterize the pharmacokinetics (PK) of GDC-0980; and (3) Evaluate preliminary evidence of antitumor activity.

Exploratory Objectives were to: (1) Evaluate changes in the pharmacodynamic marker pAKT in platelet-rich plasma (PRP) in response to GDC-0980; (2) Evaluate changes in tumor 18FDG uptake by PET in response to GDC-0980; and (3) Evaluate pretreatment tumor PIK3CA and PTEN status in relation to treatment response.

Pharmacokinetic and Pharmacodynamic Evaluation

PK: Plasma GDC-0980 concentrations were assayed by LC-MS/MS (Tandem Labs, Inc., Salt Lake City, Utah) with LLOQ 0.5 ng/mL and analyzed using noncompartmental analysis.

pAKT in PRP: Total AKT and pAKT (S473) in PRP were measured by Meso Scale Discovery (MSD) assay, and the percent change in pAKT relative to baseline was calculated.

PTEN Expression: Formalin-fixed, paraffin-embedded (FFPE) tissue sections from archival tissue were stained by IHC using a rabbit monoclonal antibody (138G6; Cell Signaling Technologies). Tumor cells were scored for PTEN expression if appropriate staining was observed in adjacent normal tissue. PTEN status may also be examined by qRT-PCR assay for mRNA levels, or chromosomal loss in a fluorescence in situ hybridization (FISH) assay.

PIK3CA mutation status: DNA was isolated from archival formalin-fixed, paraffin-embedded (FFPE) tissue. PIK3CA mutations were identified using either DxS allele-specific PCR with DxS (Manchester, UK) qRT-PCR assays or Sanger Sequencing. In the PIK3CA mutation-positive cohort in Stage 2, archival tumor tissue samples (either paraffin blocks or 10 to 15 unstained slides) or fresh tumor tissue will be analyzed for PIK3CA mutations (see Section 4.1.2.) The PIK3CA gene encodes the catalytic subunit of the PI3K protein and the frequency of PIK3CA mutations in breast cancer has been estimated at 30-40 percent. There are three mutation hotspots: E542K and E545K in exon 9 and H1047R in exon 20, which represent ≧80% of all PIK3CA mutations described. Following histopathologic review, FFPE tissue samples with ≦50% tumor content will be enriched for tumor content by macro- or micro-dissection. DNA will be isolated and analyzed in a central laboratory using a real-time PCR assay designed to detect mutations in a minimum of the three hotspots and a less common mutation in exon 7. Assays from all samples will undergo mutation detection and classification of mutation status will be performed using the manufacturer's specified protocols. This test targets eight single nucleotide mutations in four hotspots that have been found to be common in human cancer and transform cells in vitro: C420R, E542K, E545K, E545G, E545A, H1047R, H1047L, and H1047Y. Additional studies of PI3-kinase pathway-related proteins and PTEN protein expression by IHC may be performed on the collected tissue samples.

Patient Characteristics

Forty-two patients had received GDC-0980 in the study (Table 2). The baseline patient characteristics are shown in Table 2. Prior cancer therapy data for patients treated at 40 mg QD on the 28/28 daily dose schedule was not available at data cut-off.

TABLE 2

| | | |
|---|---|---|
| Median Age, yrs (range) | 58 | (35-84) |
| Gender: n (%) | | |
| Male | 25 | (59.5%) |
| Female | 17 | (40.5%) |
| ECOG performance status: n (%) | | |
| 0 | 28 | (66.7%) |
| 1 | 14 | (33.3%) |
| No. of prior systemic therapies: median | 2 | (0-10) |
| (range) | | |
| Months since primary diagnosis: median | 39 | (6-210) |
| (range) | | |
| Tumor type at initial diagnosis: n (%) | | |
| Mesothelioma | 6 | (14.3%) |
| Sarcoma | 6 | (14.3%) |
| GIST | 5 | (11.9%) |
| CRC | 5 | (11.9%) |
| Breast | 3 | (7.1%) |
| Other | 17 | (40.0%) |

Of the 42 enrolled patients in the Phase I study, six patients were on study for at least 6 months; 2 patients (peritoneal mesothelioma and adrenal cancer patients) were still on study after a year. The MTD was exceeded at 70 mg QD on a 21/28-day schedule with DLTs of Grade (G) 3 maculopapular rash and G3 hyperglycemia.

Pharmacokinetic analysis by mean plasma concentration time measurement after a single oral dose showed GDC-0980 was rapidly absorbed (Tmax about 2 hours) with a half-life of 6-18 hours and demonstrated dose-proportional increases in AUC and Cmax. Decreases in pAKT in PRP of =90% were observed at doses of 16 mg. Significant inhibition of pAKT was maintained for 24 hours at doses of 50 mg. Decreased levels of pAKT in PRP were inversely correlated with plasma GDC-0980 concentrations.

Safety

GDC-0980 was generally well tolerated through 50 mg QD administered on a 21/28 day schedule. The MTD was exceeded at 70 mg with G3 symptomatic hyperglycemia and G3 maculopapular rash. Drug-related AEs that occurred in =10% of patients (n=33) included fatigue, diarrhea, rash, nausea, decreased appetite, mucositis, hyperglycemia, vomiting, pruritus, GERD (Gastroesophageal reflux disease), and constipation. The MTD was exceeded at 70 mg QD on a 21/28 schedule with DLTs of G3 maculopapular rash and symptomatic G3 hyperglycemia. The rash resolved to G1 after ˜1 week following discontinuation of GDC-0980 and treatment with antihistamines. The patient with hyperglycemia was treated with IV hydration, insulin, and metformin. The blood glucose was within normal limits at a 30 day follow-up visit. Other toxicities in patients enrolled at 70 mg were also reversible (pneumonitis was treated with steroids and typically resolved within ˜1 week) and included:
G3 hyperglycemia and G2/3 mucositis (GDC-0980 discontinued early in Cycle 3)
G3 diarrhea, G2 pneumonitis (GDC-0980 dose reduced to 50 mg; pt still on study)
G3 pneumonitis (GDC-0980 discontinued end of Cycle 3)
G2 pneumonitis (GDC-0980 discontinued end of Cycle 2)

One G5 adverse event of colitis occurred and was considered related to GDC-0980. A patient with metastatic colorectal cancer was admitted to the hospital with severe diarrhea about 1 week after starting Cycle 2 of 50 mg QD GDC-0980. The patient experienced multiple complications while hospitalized, including a bowel perforation, pneumonia, and sepsis resulting in death approximately 3 weeks after being admitted to the hospital.

Fasted blood glucose & insulin were monitored at clinical visits (pre-dose & 2 hrs post-dose). Dosing with GDC-0980 was allowed with =G2 blood glucose with appropriate monitoring, i.e., additional monitoring in clinic and fingerstick glucose testing at home. The initiation of oral anti-diabetic agents (OAD) was suggested for =G2 blood glucose. Increased blood glucose occurred in 83.3% of the patients. Six patients experienced a G3 increased blood glucose.

Clinical Activity

Five of six patients with evaluable ¹⁸FDG-PET scans had a 25% decrease in FDG avidity by PET in either Cycle 1 or Cycle 2 dosing of GDC-0980. There were no PIK3CA hotspot mutations identified in these 6 patients and 1 patient was PTEN negative (sarcoma cancer patient) based on analysis of archival tumor tissue.

The Mean % Change from Baseline in SUVmax in Best 18FDG-PET Response by Patient was measured over time. Patients with neuro-endocrine, giant cell sarcoma, and GIST (Gastrointestinal stromal tumor) showed positive responses from 36 to 299 days at doses ranging from 16 to 70 mg daily.

Best RECIST response by evaluable patient was measured. Negative change from baseline in target lesions were measured for cancer types: RCC (Renal cell carcinoma), GIST (Gastrointestinal stromal tumor), NSCL (Non-small cell lung), Adrenal, Epithelial mesothelioma, Thyroid, Cervical, Breast, CRC, Epitheloid mesothelioma, peritoneal mesothelioma, and Adrenal cortical. Tissue analysis of RECIST response patient with Epitheloid mesothelioma showed at least one PIK3CA mutant R88Q.

Outcome Measures

The primary efficacy outcome measure is objective response (defined as a complete or partial response determined on two consecutive occasions ≧4 weeks apart), as assessed through independent radiologic review using Response Evaluation Criteria for Solid Tumors (RECIST).

The secondary efficacy outcome measures are: (1) Duration of objective response, as assessed through independent radiologic review using RECIST; (2) Overall survival and progression free survival (PFS), as assessed through independent radiologic review using RECIST; (3) CBR based on independent radiologic review using RECIST; (4) Objective response based on investigator assessments using RECIST; (5) Duration of objective response based on investigator assessments using RECIST; (6) PFS based on investigator assessments using RECIST, and (7) Tumor marker decreases (CA125), although not typically a trial endpoint, may be construed as a sign of activity.

The safety outcome measures are: (1) Incidence of adverse events and serious adverse events; (2) Incidence, nature, and relatedness of serious adverse events; (3) Incidence of adverse events leading to GDC-0980 discontinuation, modification, or interruption; Incidence and magnitude of declines in left ventricular ejection fraction (LVEF); (4) Incidence of symptomatic congestive heart failure (CHF); and (5) Cause of death on study.

The pharmacokinetic outcome measures are: (1) Serum concentrations of total trastuzumab and GDC-0980; and (2) Plasma concentrations of free DM1.

Efficacy Analyses

No adjustments for multiplicity of endpoints or within-subgroup comparisons will be incorporated in the efficacy analyses. The primary analysis population will be based on the treated population, which is defined as patients who received at least one dose of study drug. In addition, as a sensitivity analysis, the primary endpoint will be assessed in the efficacy-evaluable population, which is defined as patients who receive at least one dose of study drug and undergo at least one post-baseline response assessment, which includes, at a minimum, an assessment of all target lesions, or who die while on therapy. The secondary and exploratory efficacy analyses will be performed on the efficacy-evaluable population. The primary efficacy endpoint of this study is objective response, as assessed by independent radiologic review using RECIST. Objective response is defined as a complete or partial response determined on two consecutive occasions ≧4 weeks apart. An estimate of the objective response rate and 95% confidence intervals (Blyth-Still-Casella) will be calculated. The primary analysis population will be based on the treated population; for this analysis, patients without at least one post-baseline response assessment will be considered non-responders. In addition, objective response rate will be assessed in the efficacy-evaluable population, which is defined as patients who receive at least one dose of study drug and undergo at least one post-baseline response assessment, which includes, at a minimum, an assessment of all target lesions, or who die while on therapy.

Duration of objective response was assessed for patients with an objective response. Duration of objective response is defined as the time from the initial documentation of response to documented disease progression (including isolated CNS progression) or death from any cause on study. Separate analyses of duration of objective response will be performed based on IRF and investigator assessments. Methods for handling censoring for analysis are the same as described below for PFS.

Progression-Free Survival (PFS) is defined as the time from the first day of treatment to documented disease progression (including isolated CNS progression) or death from any cause on study, whichever occurs first. Death on study is defined as death from any cause within 30 days of the last dose of GDC-0980. Separate analyses of PFS will be performed based on IRF and investigator assessments. PFS will be estimated for efficacy-evaluable patients only. PFS data for patients without disease progression or death will be censored at the time of the last tumor assessment. Kaplan-Meier estimates of median PFS and PFS rates at 6 and 9 months will be reported as appropriate.

Clinical Benefit Rate (CBR) is defined as the proportion of patients with a complete or partial response or stable disease at 6 months. Patients without at least one post-baseline response assessment will be considered as experiencing no clinical benefit. CBR will be calculated separately for tumor assessments based on investigator and an IRF assessment.

Safety Analyses: All patients who receive any amount of GDC-0980 therapy will be included in the safety analyses. Safety will be assessed through summaries of adverse events, deaths, and changes in laboratory test results. All adverse events for all patients will be collected for the safety dataset. Verbatim descriptions of adverse events will be mapped to thesaurus terms. All recorded adverse event data will be listed by study site, patient, and cycle. All adverse events occurring on or after the first treatment will be summarized by mapped term, appropriate thesaurus levels, and NCI CTCAE, v3.0 toxicity grade. All serious adverse events will be listed separately and summarized. In addition, the incidence of symptomatic CHF and/or LVEF <40% will be summarized. Deaths reported during the study treatment period and those reported during follow-up after patient treatment discontinuation will be summarized. Laboratory data will be summarized by grade using the NCI CTCAE, v3.0 toxicity grade. Changes in LVEF over time will be summarized and listed by scheduled measurement time. The occurrence of antibodies to GDC-0980 will be listed.

Pharmacokinetic and Pharmacodynamic Analyses: For GDC-0980 descriptive statistics, including mean and median trough and peak values, will be summarized. The following PK parameters will be estimated following the first through fourth doses and every other dose thereafter: AUC, maximum serum concentration, CL, volume of distribution, and half-life.

Patient-Reported Outcome Assessments: The FACT-B, as well as the FACT-B subscale (TOI-PFB), and Patient's Assessment of Pain will be used to explore the impact of GDC-0980 on patient-reported symptoms. Mean scores and change from baseline to each timepoint will be assessed for all efficacy-evaluable patients, responders, and patients with stable disease or who are non-responders. The proportion of patients who have a clinically significant change in TOI-PFB scores at each timepoint will also be assessed. A change of 5 points in the TOI-PFB score is considered clinically significant.

Differences in Symptoms in Clinical Responders and Non-Responders: As an exploratory endpoint, differences in symptom progression between clinical responders and non-responders will be compared. Patients for whom no baseline TOI-PFB score is available, or for whom no post-baseline TOI-PFB score is available, will be excluded from this analysis.

Missing Data: For objective response, patients without a post-baseline tumor assessment will be considered non-responders. For duration of response and PFS, data from patients who are lost to follow-up will be included in the analysis as censored observations on the last date that the patient was known to be progression-free, defined as the date of the last tumor assessment. Determination of Sample Size: This study was designed to determine the efficacy and safety of GDC-0980 in patients with solid tumors and NHL.

Statistical Methods

Continuous data was summarized using mean, standard deviation, median, minimum, and maximum. Discrete data will be summarized using frequencies and percentages.

### Example 3

### Assessments During Treatment

Three patients with mesothelioma and a patient with adrenal cortical cancer showed significant tumor shrinkage by RECIST:

A 55-year-old woman, diagnosed with adrenal cortical cancer in 2004, was previously treated with surgery for extensive metastatic disease in the liver, pelvic wall, and peritoneum. The patient enrolled at 40 mg QD GDC-0980 (AUC0-24 h ˜4.1 μM·hr) on a 28/28 day schedule and demonstrated a 22% decrease in target lesions by RECIST at the end of Cycle 1 and 39% decrease after 3 weeks of treatment in Cycle 2. GDC-0980 was interrupted in Cycle 2 due to adverse events of Grade 2 increased ALT and Grade 3 rash. The patient remained on study at a reduced dose of GDC-0980. The right subhepatic lesion is shown in PET images.

A 60-year-old man, diagnosed with epitheloid mesothelioma cancer four years earlier, was previously treated with XRT and cisplatin/pemetrexed. Analysis of archival tumor tissue showed a R88Q mutation in PIK3CA exon 2. The patient enrolled at 8 mg QD GDC-0980 (AUC0-24 h ˜0.4 μM·hr) on a 21/28 day schedule and demonstrated a 26% decrease in target lesions by RECIST at the end of Cycle 2. The patient was on study for about 4.5 months before progression of disease. The mediastinal disease is shown in PET images.

A 32-year-old woman, diagnosed with peritoneal mesothelioma cancer seven years earlier, was previously treated with XRT and cisplatin/pemetrexed. The patient enrolled at 32 mg QD GDC-0980 (AUC0-24 h ˜4.4 μM·hr) on a 21/28 day schedule and demonstrated a 28% decrease in target lesions by RECIST at the end of Cycle 6. The patient is currently on study after 1 year 3 months. A peritoneal nodule in the left iliac fossa is shown in PET images.

### Example 4

### Response Evaluation Criteria in Solid Tumors (RECIST)

The Response Evaluation Criteria in Solid Tumors (RECIST) was applied according to: Therasse P, Arbuck S G, Eisenhauser E A, Wanders J, Kaplan R S, Rubinstein L, et al. New guidelines to evaluate the response to treatment in solid tumors. J Natl Cancer Inst 2000; 92:205-16); and as follows.

Measurability Of Tumor Lesions At Baseline: At baseline, tumor lesions will be categorized as follows: measurable (lesions that can be accurately measured in at least one dimension [longest diameter to be recorded] as 20 mm with conventional techniques or as 10 mm with spiral CT scan) or nonmeasurable (all other lesions, including small lesions [longest diameter <20 mm with conventional techniques or <10 mm with spiral CT scan] and truly nonmeasurable lesions). The term “evaluable” in reference to measurability is not recommended and will not be used because it does not provide additional meaning or accuracy. All measurements should be recorded in metric notation by use of a ruler or calipers. All baseline evaluations should be performed as closely as possible to the beginning of treatment and never more than 4 weeks before the beginning of treatment. Lesions considered to be truly nonmeasurable include the following: bone lesions, leptomeningeal disease, ascites, pleural/pericardial effusion, inflammatory breast disease, lymphangitis cutis/pulmonis, abdominal masses that are not confirmed and followed by imaging techniques, and cystic lesions. Tumor lesions that are situated in a previously irradiated area might or might not be considered measurable, and the conditions under which such lesions should be considered must be defined in the protocol when appropriate.

Specifications by Methods of Measurements: The same method of assessment and the same technique should be used to characterize each identified and reported lesion at baseline and during follow-up. Imaging-based evaluation is preferred to evaluation by clinical examination when both methods have been used to assess the antitumor effect of a treatment.

Clinical Examination Clinically detected lesions will only be considered measurable when they are superficial (e.g., skin nodules and palpable lymph nodes). For the case of skin lesions, documentation by color photography—including a ruler to estimate the size of the lesion—is recommended.

Chest X-ray: Lesions on chest X-rays are acceptable as measurable lesions when they are clearly defined and surrounded by aerated lung. However, CT is preferable. More details concerning the use of this method of assessment for objective tumor response evaluation are provided in Therasse P, Arbuck S G, Eisenhauser E A, Wanders J, Kaplan R S, Rubinstein L, et al. New guidelines to evaluate the response to treatment in solid tumors. (2000) J Natl Cancer Inst 92:205-16.

CT and MRI: CT and MRI are the best currently available and most reproducible methods for measuring target lesions selected for response assessment. Conventional CT and MRI should be performed with contiguous cuts of 10 mm or less in slice thickness. Spiral CT should be performed by use of a 5-mm contiguous reconstruction algorithm; this specification applies to the tumors of the chest, abdomen, and pelvis, while head and neck tumors and those of the extremities usually require specific protocols.

Ultrasound: When the primary endpoint of the study is objective response evaluation, ultrasound should not be used to measure tumor lesions that are clinically not easily accessible. It may be used as a possible alternative to clinical measurements for superficial palpable lymph nodes, subcutaneous lesions, and thyroid nodules. Ultrasound might also be useful to confirm the complete disappearance of superficial lesions usually assessed by clinical examination.

Endoscopy and Laparoscopy: The utilization of these techniques for objective tumor evaluation has not yet been fully or widely validated. Their uses in this specific context require sophisticated equipment and a high level of expertise that may be available only in some centers. Therefore, utilization of such techniques for objective tumor response should be restricted to validation purposes in specialized centers. However, such techniques can be useful in confirming complete histopathologic response when biopsy specimens are obtained.

Tumor Markers: Tumor markers alone cannot be used to assess response. However, if markers are initially above the upper normal limit, they must return to normal levels for a patient to be considered in complete clinical response when all tumor lesions have disappeared. Specific additional criteria for standardized usage of prostate-specific antigen and CA (cancer antigen) 125 response in support of clinical trials are being validated.

Cytology and Histology: Cytologic and histologic techniques can be used to differentiate between partial response and complete response in rare cases (e.g., after treatment to differentiate between residual benign lesions and residual malignant lesions in tumor types such as germ cell tumors). Cytologic confirmation of the neoplastic nature of any effusion that appears or worsens during treatment is required when the measurable tumor has met criteria for response or stable disease. Under such circumstances, the cytologic examination of the fluid collected will permit differentiation between response and stable disease (an effusion may be a side effect of the treatment) and progressive disease (if the neoplastic origin of the fluid is confirmed). New techniques to better establish objective tumor response will be integrated into these criteria, when they are fully validated, to be used in the context of tumor response evaluation.

Tumor Response Evaluation and Assessment of Overall Tumor Burden and Measurable Disease: To assess objective response, it is necessary to estimate the overall tumor burden at baseline to which subsequent measurements will be compared. Only patients with measurable disease at baseline should be included in protocols where objective tumor response is the primary endpoint. Measurable disease is defined by the presence of at least one measurable lesion. If the measurable disease is restricted to a solitary lesion, its neoplastic nature should be confirmed by cytology/histology.

Baseline Documentation of “Target” and “Nontarget” Lesions: All measurable lesions up to a maximum of 5 lesions per organ and 10 lesions in total, representative of all involved organs, should be identified as target lesions and recorded and measured at baseline. Target lesions should be selected on the basis of their size (those with the longest diameter) and their suitability for accurate repeated measurements (either by imaging techniques or clinically). A sum of the longest diameter for all target lesions will be calculated and reported as the baseline sum longest diameter. The baseline sum longest diameter will be used as the reference by which to characterize the objective tumor response. All other lesions (or sites of disease) should be identified as nontarget lesions and should also be recorded at baseline. Measurements of these lesions are not required, but the presence or absence of each should be noted throughout follow-up.

Response Criteria and Evaluation of Target Lesions: Criteria have been adapted from the original WHO Handbook, taking into account the measurement of the longest diameter only for all target lesions: complete response—the disappearance of all target lesions; partial response—at least a 30% decrease in the sum of the longest diameter of target lesions, taking as reference the baseline sum longest diameter; progressive disease—at least a 20% increase in the sum of the longest diameter of target lesions, taking as reference the smallest sum longest diameter recorded since the treatment started or the appearance of one or more new lesions; stable disease—neither sufficient shrinkage to qualify for partial response nor sufficient increase to qualify for progressive disease, taking as reference the smallest sum longest diameter since the treatment started.

Evaluation of Nontarget Lesions: The definitions of the criteria used to determine the objective tumor response for nontarget lesions include: complete response—the disappearance of all nontarget lesions and normalization of tumor marker level; incomplete response/stable disease—the persistence of one or more nontarget lesion(s) and/or the maintenance of tumor marker level above the normal limits; and progressive disease—the appearance of one or more new lesions and/or unequivocal progression of existing nontarget lesions.

IRF—Independent Review Facility; INV—Investigator; Objective Response—CR or PR determined by two consecutive tumor assessments at least 28 days apart; Clinical Benefit—objective response or SD maintained for at least 6 months

Evaluation of Best Overall Response: The best overall response is the best response recorded from the start of treatment until disease progression/recurrence (taking as reference for progressive disease the smallest measurements recorded since the treatment started). In general, the patient's best response assignment will depend on the achievement of both measurement and confirmation criteria.

Table 3 provides overall responses for all possible combinations of tumor responses in target and nontarget lesions with or without the appearance of new lesions.

TABLE 3

| | | | Overall |
|---|---|---|---|
| Target Lesions | Nontarget Lesions | New Lesions | Response |
| CR | CR | No | CR |
| CR | Incomplete response/SD | No | PR |
| PR | Non-PD | No | PR |
| SD | Non-PD | No | SD |
| PD | Any | Yes or No | PD |
| Any | PD | Yes or No | PD |
| Any | Any | Yes | PD |

* CR = complete response; PR = partial response; SD = stable disease; and PD = progressive disease.
a. Patients with a global deterioration of health status requiring discontinuation of treatment without objective evidence of disease progression at that time should be classified as having “symptomatic deterioration.” Every effort should be made to document the objective disease progression, even after discontinuation of treatment.
b. Conditions that may define early progression, early death, and inevaluability are study specific and should be clearly defined in each protocol (depending on treatment duration and treatment periodicity).
c. In some circumstances, it may be difficult to distinguish residual disease from normal tissue. When the evaluation of complete response depends on this determination, it is recommended that the residual lesion be investigated (fine-needle aspiration/biopsy) before confirming the complete response status.

Frequency of Tumor Re-Evaluation: Frequency of tumor re-evaluation while on treatment should be protocol specific and adapted to the type and schedule of treatment. However, in the context of Phase II studies where the beneficial effect of therapy is not known, follow-up of every other cycle (i.e., 6 to 8 weeks) seems a reasonable norm. Smaller or greater time intervals than these could be justified in specific regimens or circumstances. After the end of the treatment, the need for repetitive tumor evaluations depends on whether the Phase II trial has, as a goal, the response rate, or the time to an event (disease progression/death). If time to an event is the main endpoint of the study, then routine re-evaluation is warranted of those patients who went off the study for reasons other than the expected event at frequencies to be determined by the protocol. Intervals between evaluations twice as long as on study are often used, but no strict rule can be made.

Confirmatory Measurement/Duration of Response: The main goal of confirmation of objective response in clinical trials is to avoid overestimating the response rate observed, useful in nonrandomized trials where response is the primary endpoint. In this setting, to be assigned a status of partial response or complete response, changes in tumor measurements must be confirmed by repeat assessments that should be performed no less than 4 weeks after the criteria for response are first met. Longer intervals as determined by the study protocol may also be appropriate. In the case of stable disease, measurements must have met the stable disease criteria at least once after study entry at a minimum interval (in general, not less than 6 to 8 weeks) that is defined in the study protocol.

### Example 5

### Modified RECIST Criteria for Assessment of Response in Malignant Pleural Mesothelioma

Modified RECIST criteria for assessment of response in malignant pleural mesothelioma were adopted (Byrne M J and Nowak A K. “Modified RECIST criteria for assessment of response in pleural mesothelioma”. Annals of Oncology (2004) 15:257-260).

Pleural Unidimensional Measurement: The sum of six measurements define a pleural unidimensional measure. Tumor thickness perpendicular to the chest wall or mediastinum should be measured in two positions at three separate levels on transverse cuts of CT scan. Transverse cuts at least 1 cm apart and related to anatomical landmarks in the thorax to allow reproducible assessment at later time points should be chosen. If measurable tumor is present above the level of the division of the main bronchi measurement of tumor in transverse cuts of the upper thorax are preferable. At reassessment, pleural thickness is to be measured at the same position at the same level and by the same observer. Note this will not necessarily be the greatest tumor thickness at that level.

Total Tumor Measurement: Nodal, subcutaneous and other bidimensionally measurable lesions should be measured unit dimensionally as per the RECIST criteria Example 7.

Response Criteria Complete response (CR) is defined as the disappearance of all target lesions with no evidence of tumor elsewhere. Partial response (PR) is defined as at least a 30% reductions in the total tumor measurement. For both CR and PR a confirmed response requires a repeat observation on two occasions 4 weeks apart. Progressive disease (PD) is defined as an increase of at least 20% in the total tumor measurement over the nadir measurement, or the appearance of one or more new lesions. Patients with stable disease (SD) are those that do not fulfill the criteria for CR, PR or PD.

The foregoing description is considered as illustrative only of the principles of the invention. Further, since numerous modifications and changes will be readily apparent to those skilled in the art, it is not desired to limit the invention to the exact construction and process shown as described above. Accordingly, all suitable modifications and equivalents may be considered to fall within the scope of the invention as defined by the claims that follow.

We claim:

1. A method for the treatment of mesothelioma comprising administering to a patient with mesothelioma a therapeutically effective amount of GDC-0980 having the structure:

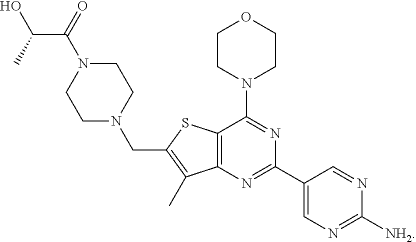

2. The method of claim 1, wherein the patient has malignant pleural mesothelioma.

3. The method of claim 1, wherein the patient has been previously treated with chemotherapy, radiotherapy, and/or surgical resection.

4. The method of claim 3, wherein the patient has been previously treated with one or more chemotherapeutic agents selected from pemetrexed, bevacizumab, cisplatin, gemcitabine, vinorelbine, imatinib, dasatinib, erlotinib, sunitinib, and sorafenib.

5. The method of claim 1, wherein GDC-0980 is administered daily at three week or four week intervals to the patient.

6. The method of claim 5 wherein the three week interval is followed by a one week holiday interval where the patient is not administered GDC-0980.

7. The method of claim 1, wherein GDC-0980 is administered orally.

8. The method of claim 1, wherein the therapeutically effective amount of GDC-0980 is 1 mg to 100 mg per day of patient body weight.

9. The method of claim 1, wherein the therapeutically effective amount of GDC-0980 is 10 mg to 50 mg per day of patient body weight.

10. The method of claim 1, further comprising administering a chemotherapeutic agent selected from selected from pemetrexed, bevacizumab, cisplatin, gemcitabine, vinorelbine, imatinib, dasatinib, erlotinib, sunitinib, or sorafenib.

11. The method of claim 1, wherein GDC-0980 is formulated with an ingredient selected from microcrystalline cellulose, lactose monohydrate, croscarmellose sodium, copovidone, and magnesium stearate.

12. The method of claim 1, wherein GDC-0980 is formulated with a pharmaceutically acceptable glidant selected from silicon dioxide, powdered cellulose, microcrystalline cellulose, metallic stearates, sodium aluminosilicate, sodium benzoate, calcium carbonate, calcium silicate, corn starch, magnesium carbonate, asbestos free talc, stearowet C, starch, starch 1500, magnesium lauryl sulfate, magnesium oxide, and combinations thereof.
